(19) United States
(12) Patent Application Publication
(10) Pub. No.: US 2016/0010077 A1
(43) Pub. Date: Jan. 14, 2016

(54) Covalent Attachment of Bacteriophages (Phages) to Polymeric Surfaces

(71) Applicant: THE UNIVERSITY OF SOUTHERN MISSISSIPPI, Hattiesburg, MS (US)

(72) Inventors: Marek W. Urban, Clemson, SC (US); Mohamed o Elasri, Hattiesburg, MS (US)

(73) Assignee: The University of Southern Mississippi, Hattiesburg, MS (US)

(21) Appl. No.: 14/771,439

(22) PCT Filed: Feb. 27, 2014
(86) PCT No.: PCT/US14/19003

§ 371 Date: Aug. 28, 2015

Related U.S. Application Data
(60) Provisional application No. 61/770,422, filed on Feb. 28, 2013.

(51) Int. Cl. C12N 11/06

(52) U.S. Cl. CPC C12N 11/06

(57) ABSTRACT

We disclose a method of covalently attaching bacteriophages to a surface, including polymers, to create a resulting antibacterial surface device. Because the bacteriophages are specific for bacteria, other organisms for which the phages are not specific are not damaged by the phage-modified surfaces.

### CROSS-REFERENCES TO RELATED APPLICATIONS

This application is the 35 U.S.C. §371 national stage application of International Patent Application No. PCT/2014US/019003, which claims the benefit of U.S. Provisional Application No. 61/770,422, filed Feb. 28, 2013, both of which are incorporated herein by reference in their entirety.

### STATEMENT REGARDING FEDERALLY SPONSORED RESEARCH OR DEVELOPMENT

This invention was made with government support under 2002-2010 MRSEC DMR 0213883, awarded by the National Science Foundation, and ONR N00014-07-1-1057, awarded by the Office of Naval Research. The government has certain rights in the invention.

### FIELD OF THE INVENTION

The present invention is generally directed toward a method of covalent attachment of bacteriophages to surfaces, including polymeric surfaces, and resulting anti-bacterial surface devices.

### BACKGROUND

Although, the majority of interactions between biologically active species and synthetic materials are inherently unfavorable, there are some exceptions. For example, the formation of microbial biofilms that often leads to detrimental consequences is an undesirable, but readily occurring, process that has become a serious medical problem. Structurally simple and smaller, as compared to the vast majority of eukaryotic cells, bacteria display a wide array of surface appendages capable of feeding from other organisms and are, therefore, capable of the growth and adhesion to a variety of surfaces. As a consequence, microbial films are formed.

Although antibiotics are the primary line of defense against bacterial infections, the number of fatalities resulting from the inability of these drugs to defeat microbial films is rising. The main strategy to alleviate this growing problem is the development of new drugs. In spite of these efforts, bacterial mutations and biofilm formation continue to be a threat.

To battle microbial film growths, there are numerous efforts to modify substrate surfaces in contact with cellular environments. Among notable advances is the covalent attachment of antibiotics or other antimicrobial agents (Aumsuwan, N., Heinhorst, S., Urban, M W. Antibacterial Surfaces on Expanded Polytetralluoroethylene (ePTFE); Penicillin Attachment. Biomacromolecules, 8, 713-718 (2007), incorporated herein by reference). This inherent resistance to host defenses and antimicrobial agents resulted in the development of novel approaches to avoid biofilm formation on medical devices and temporarily prevent implant infections, but the problem is far from being under control.

### SUMMARY OF THE INVENTION

In one aspect, methods of synthetic paths are disclosed for covalently attaching bacteriophages (phages) to surfaces, including any polymeric surface, which is accomplished by NH2-COOH reactions leading to amide linkages. During this process phages retain their biological activity manifested by a rapid destruction of bacteria. In another aspect, the resulting devices having phage-bound surfaces, including polymeric surfaces, are disclosed.

### BRIEF DESCRIPTION OF THE DRAWINGS

Further advantages of the invention will become apparent by reference to the detailed description of preferred embodiments when considered in conjunction with the drawings:

FIG. 1A depicts the attachment of phages to bacteria and injection of nucleic acid.

FIG. 1B depicts replication of nucleic acid and virion particles, then destruction of bacteria.

FIG. 2A depicts a plot showing ATR-FTIR spectra of UHMWPE surface (Trace A), after plasma reactions on UHMWPE surfaces in the presence of maleic anhydride (MA) (Trace B), after T1 phage covalent attachment to MA-UHMWPE modified surface (Trace C); and Reference spectrum of T1 phage (Trace D).

FIG. 2B depicts a plot showing ATR-FTIR spectra of PTFE surface (Trace A), after plasma reactions on PTFE surfaces in the presence of maleic anhydride (MA) (Trace B), after T1 phage covalent attachment to MA-PTFE modified surface (Trace C); Reference spectrum of T1 phage (Trace D).

FIG. 3A shows height profiles and AFM images of silicon (Si) wafer for height profile of Si wafer (A-1), height image of Si wafer (A-2), phase image of Si wafer (A-3).

FIG. 3B shows height profiles and AFM images of silicon (Si) wafer for height profile of Si plasma reacted surface exhibiting —COOH groups (B-1), height image of Si-MA (B-2), phase image of Si-MA (B-3).

FIG. 3C shows height profiles and AFM images of silicon (Si) wafer for height profile of T1 phage attached Si (C-1), height image of Si-MA-T1 phage (C-2), phase image of Si-MA-T1 phage (C-3).

FIGS. 4A-H show plaque formation assays for covalently attached T1 and Φ11 phages on PE and PTFE surfaces. FIG. 4A shows PE-T1 phage surface in E. coli plate. FIG. 4B shows PTFE-T1 phage surface in E. coli plate. FIG. 4C shows PE-T1/Φ11 mixed phage surface in E. coli plate. FIG. 4D shows PTFE-T1/Φ11 mixed phage surface in E. coli plate. FIG. 4E shows PE-Φ11 phage surface in S. aureus plate. FIG. 4F shows PTFE-Φ11 phage surface in S. aureus plate. FIG. 4G shows PE-T1/Φ11 mixed phage surface in S. aureus plate. FIG. 4H shows PTFE-T1/Φ11 mixed phage surface in S. aureus plate.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

Using our methods disclosed herein, ability of viruses to destroy bacteria can be harnessed. Unlike bacteria, viruses rely on the “hospitality” of a host to replicate themselves, or may remain dormant if no living hosts are available. In essence, viruses comprise of fragmented nucleic acid sequences with encoded instructions for replication, enclosed within a protein shell or a membrane. Unlike bacteria, viruses exhibit mono-dispersed sizes that range from a few angstroms to microns. Furthermore, each type of virus has its own specific shape.

Some viruses have the ability to recognize specific bacteria and infect them by anchoring to the bacterial surface, injecting the viral genetic material into the bacteria, and replicating using components of the bacteria. The host bacteria are destroyed in the process. These viruses that infect and destroy bacteria are referred to as bacterial phages, bacteriophages, or phages interchangeably herein (Twort F W. An investigation on the nature of the ultramicroscopic viruses. Lancet; 189, 1241-3 (1915), incorporated herein by reference; D'Herelle F. Sur un microbe invisible antagoniste des bac. dysentériques. Crit. Rev. Acad. Sci. Paris, 165, 373, (1917), incorporated herein by reference), and are quite distinct from the animal or plant viruses.

Beginning at the onset of the 20th Century, phages were utilized to understand molecular aspects of genetics, the synthesis of proteins by DNA, and continue to serve as cloning vectors. This group of viruses (phages) possesses fairly unique shapes and became a subject of physical and chemical studies as fascinating mechanical objects. Similar to the use of polymeric materials for facilitating drug delivery by degradation of a polymer matrix, phages have been added into polymer membranes by simply physically mixing with, or non-covalent adsorption on, polymers (for example, Nylon) (D'Herelle F. The bacteriophage: its role in immunity. Williams and Wilkens Co./Waverly Press, Baltimore, USA, (1922), incorporated herein by reference; Chkhaidze J D., Imedashvili N E. The use of a novel biodegradable preparation capable of the sustained release of bacteriophages and ciprofloxacin, in the complex treatment of multidrug-resistant Staphylococcus aureus-infected local radiation injuries caused by exposure to Sr90. Clin Exp Dermatol. 30:23-6 (2005), incorporated herein by reference; Markoishvili K, Tsitlanadze G, Katsarava R, Morris J G Jr, Sulakvelidze A. A novel sustained-release matrix based on biodegradable poly(ester amide)s and impregnated with bacteriophages and an antibiotic shows promise in management of infected venous stasis ulcers and other poorly healing wounds. Int J Dermatol. 41:453-8 (2002), incorporated herein by reference). Although these approaches often represent the only means for effective drug delivery, controllable, on-demand release is often difficult. Taking advantage of the ability of phages to precisely recognize a host bacterium, we covalently attached phages onto polymeric surfaces. Thus, we disclose herein a method for the attachment of bacteriophages to polymer surfaces and the resulting anti-bacterial devices.

Methods

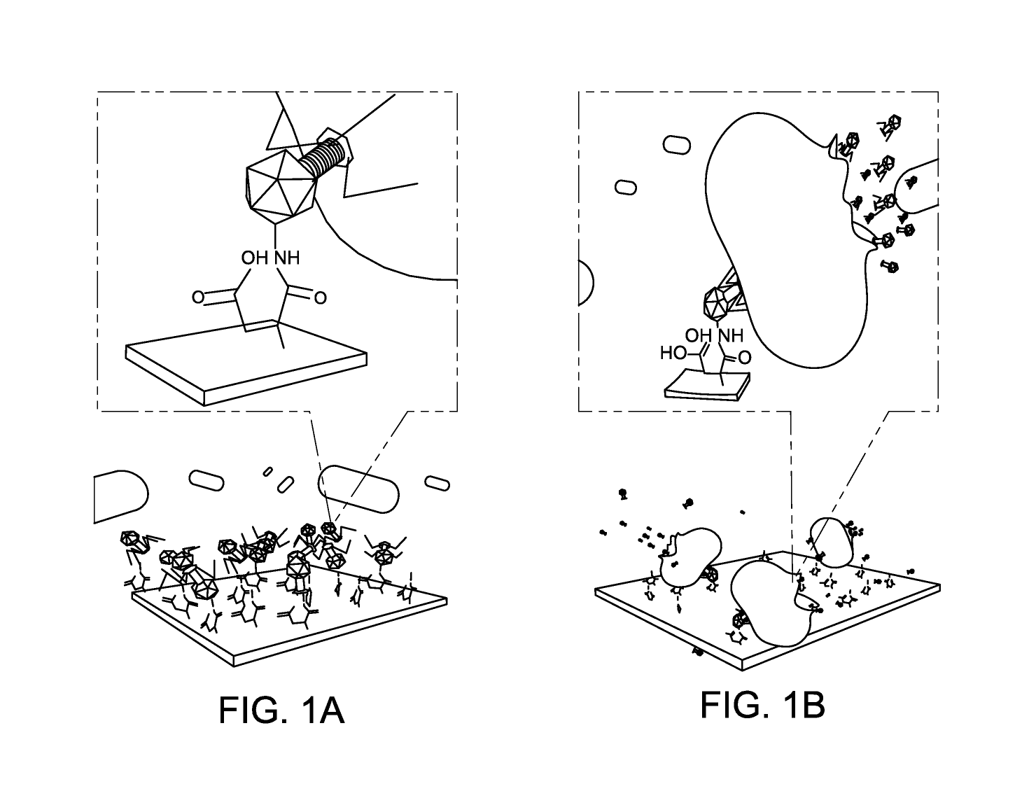

By attaching bacteriophages to the surface material, the surface material becomes deadly to bacteria. When the bacteria attempt to grow on the surface of the phage-modified substrate surface material, the phage will attach to the bacteria to which it is specific as can be seen from FIG. 1A. Any surface material suitable for exposing a carboxylic acid group thereon or reacting with a carboxylic acid group containing compound to covalently attach a carboxylic acid group to that surface material is contemplated to be within the scope of the inventions disclosed herein, e.g., silicon wafer, polymeric, plastic, or organic polymer surfaces. The attachment occurs through interactions of the distal ends of the phage tails with usually one of a plethora of cell-surface components. As a result of strong binding between the phage and the binding site on the bacterium, the phage genetic material contained within its capsid is injected into the host cell. Once within the bacteria, the phage genetic material is translated into protein, and the phage takes over the bacteria by subverting it into making new phages, thus causing disintegration (lysis) of the host bacterium. Covalent binding of all suitable bacteriophages, non-enveloped or enveloped, using the methods disclosed herein are within the contemplation of the invention, but only T1 (non-enveloped) and Φ11 (enveloped) phages with different bacteria-specificity are described in detail for sake of brevity.

Before we verified that the processes as depicted in FIGS. 1A & 1B were effective in microbial film obliterations, we conducted a series of experiments in which we visually and quantitatively assessed T1 and Φ11 attachments to the surfaces.

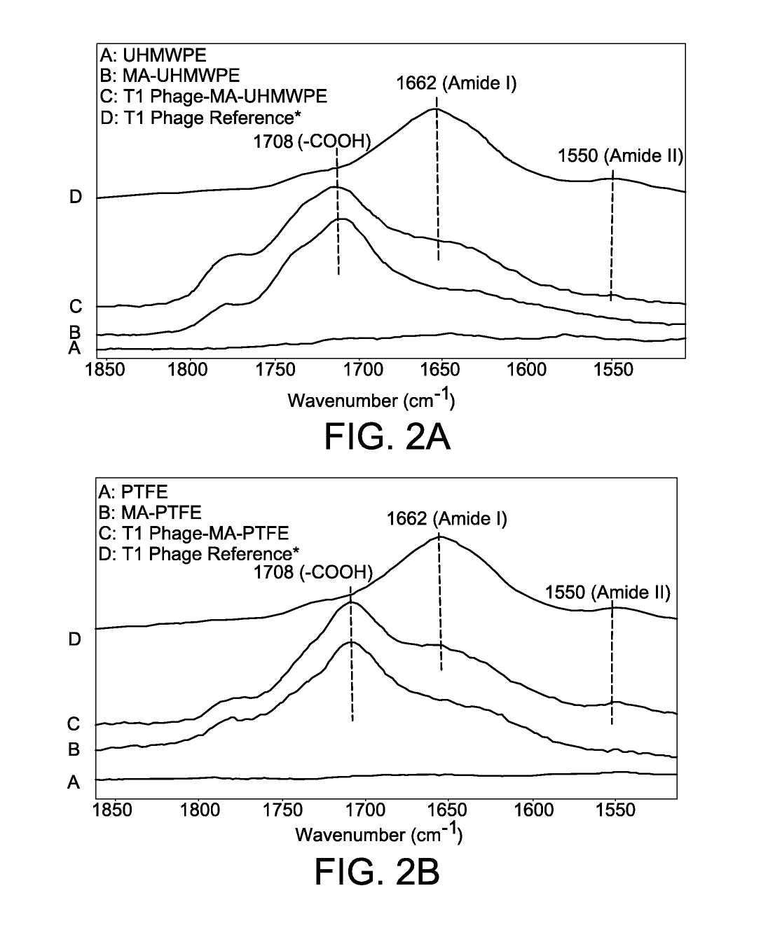

Shown in FIGS. 2A & 2B are plots illustrating ATR-FTIR spectra recorded from PE (FIG. 2A) and PTFE (FIG. 2B) surfaces (represented by Traces A), maleic anhydride plasma modified (FIG. 2A) and PTFE (FIG. 2B) surfaces (Traces B), and T1 phage covalently attached to (FIG. 2A) and PTFE (FIG. 2B) surfaces (Traces C). Traces B and C show a characteristic band at 1708 cm⁻¹ due to the —COOH modification of the polymer surface. Note that Traces A in FIGS. 2A & 2B illustrate ATR-FTIR spectra of unmodified polymer. These figures also show two characteristic bands at 1662 and 1550 cm⁻¹ due to Amide I and II bands' characteristics of the T1 phage outer functionalities. For comparison, Traces D in FIGS. 2A & 2B illustrate ATR-FTIR spectra of T1 phage alone. Similar results were obtained with Φ11 phage attachment to PE and PTFE surfaces (not shown), and with silicon (Si) wafer surfaces with MA modification and T1 or Φ11 phages attachment (not shown).

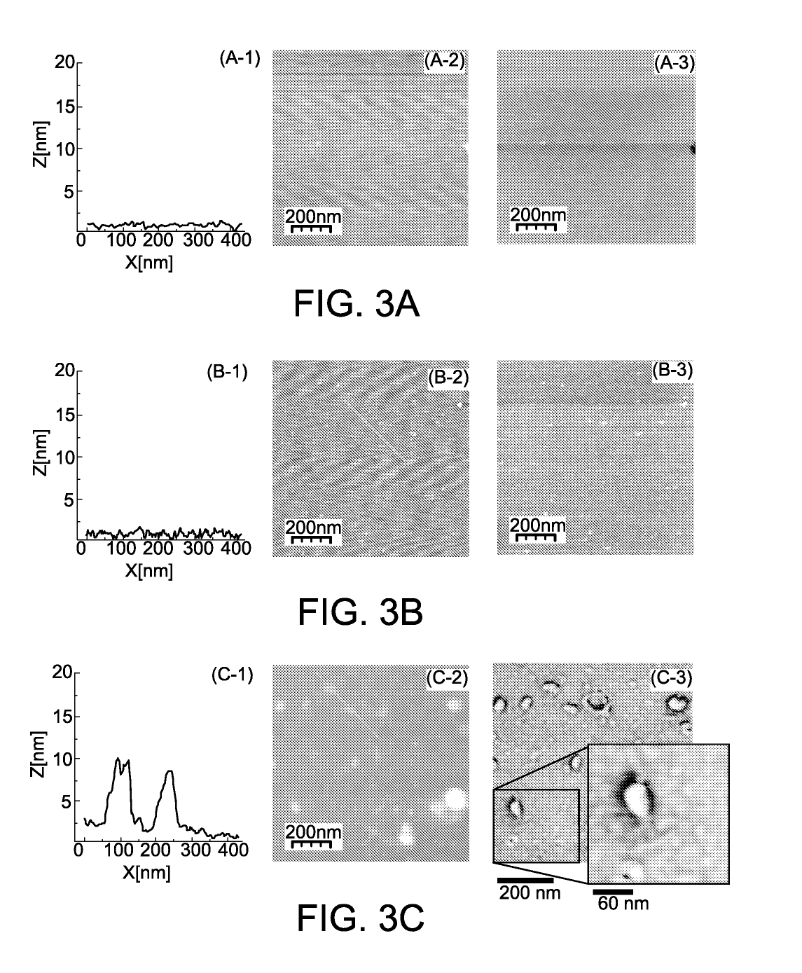

To visually assess the presence of T1 phages on surfaces, atomic force microscopy (AFM) images were collected after each step illustrated in FIG. 1A and FIG. 1B, as well as control images of unmodified surface (data for Si wafer surface is shown). As will be appreciated from FIGS. 3A, 3B, and 3C illustrate AFM data collected from silicon (Si) wafer surfaces before and during the process steps. FIG. 3A shows a profile height data plot (A-1), height image (A-2), and phase image (A-3) of the Si wafer surface before maleic anhydride treatment. FIG. 3B shows a profile height data plot (B-1), height image (B-2), and phase image (B-3) of the Si wafer surface after maleic anhydride treatment. FIG. 3C shows a profile height data plot (C-1), height image (C-2), and phase image (C-3) of the Si wafer surface after covalent attachment of T1 phages.

Comparison of height profiles in FIGS. 3B and 3C (inserts B-1 to C-1) clearly shows that, when T1 phages are reacted to the COOH-terminated surface, the surface maximum height is ˜10 nm, whereas the width is approximately 60-80 nm. The corresponding AFM images shown in FIG. 3C (inserts C-2 and C-3) visually illustrate shapes that correspond to T1 phages images reported by others and well-known in the art. Similar results were obtained for Φ11 phage modified Si wafers (not shown). AFM images were also used to quantify the mean number of phages per μm² on the surfaces. The mean average number of T1 and Φ11 phages per μm² of Si wafer was found to be 5.8±1.7 and 10.8±1.0, respectively, following these procedures.

Analysis of Biological Activity of Covalently Attached Phages

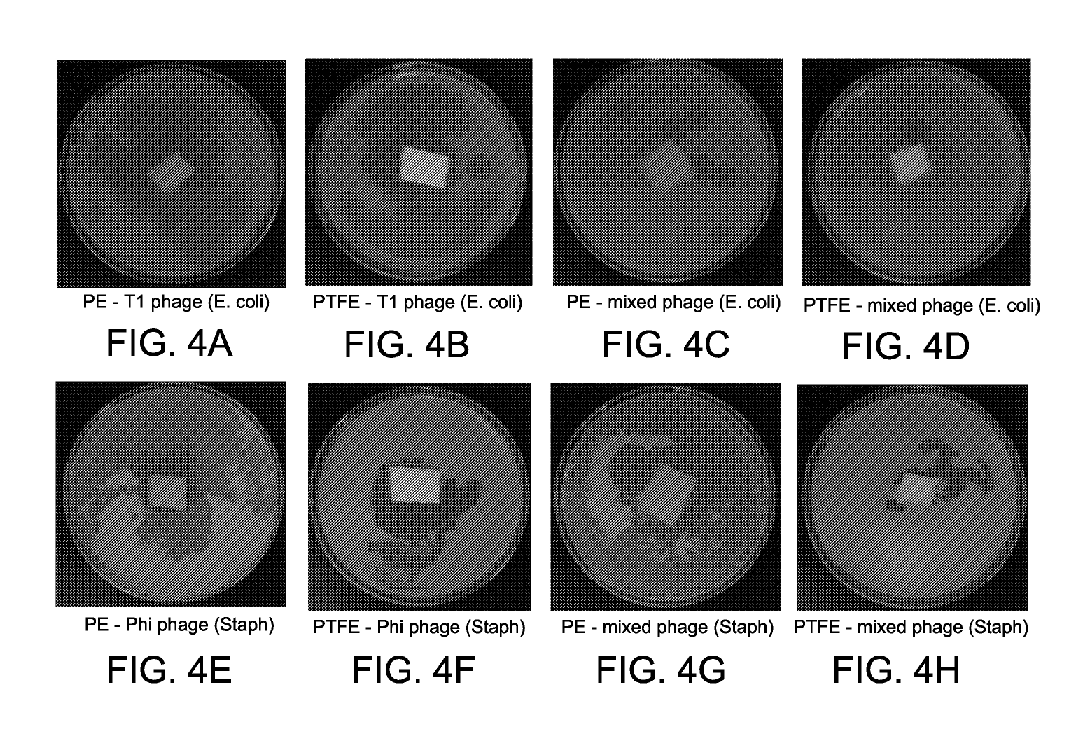

Biological activity of phages covalently attached to PTFE and PE surfaces was confirmed through the use of plaque formation assays. These assays demonstrate the selectivity of T1 and Φ11 phages for Escherichia coli and Staphylococcus aureus, respectively. FIGS. 4A & 4B show PE and PTFE surfaces, respectively, with covalently attached T1 phage in petri dishes containing a lawn of E. coli bacteria. The clear zone surrounding the polymer surfaces demonstrate that the covalently bound phages are effective in killing (lysing) bacteria. Similarly, FIGS. 4E & 4F show the same PE and PTFE surfaces, respectively, with covalently attached Φ11 phages that kill (lyse) S. aureus bacteria, also observed as the clear zone surrounding the polymer surface. Additionally, PE and PTFE surfaces were reacted with a 1:1 mixture of T1 and Φ11 phages to obtain dual phage containing surfaces. The reactivity of these surfaces against bacteria are illustrated in FIGS. 4C & 4D for PE and PTFE, respectively, with T1 and Φ11 phages against E. coli bacteria. FIGS. 4G & 4H demonstrate the effectiveness of PE and PTFE, respectively, with T1 and Φ11 phages against S. aureus bacteria. Negative controls of MA-modified and unmodified PE and PTFE without covalently attached T1 and Φ11 phages showed no plaque formation (not shown). T1 and Φ11 phages were added to separate plates as positive controls for E. coli and S. aureus, respectively. Similar results were obtained with T1 and Φ11 phages covalently attached to Si wafers (not shown).

In summary, these studies explore a universal approach of modifying surfaces using biologically active phages. These reactions can be conducted on almost any surface as long as phage biological activities are maintained. Although recent studies on stimuli-responsive materials offered a number of promising synthetic approaches to combat deadly microbial film formation, the use of phages to kill human pathogens anchored to synthetic material surfaces shows a promising method for combating antibiotic resistant infections. There are multiple possibilities of surface modifications using solitary phage or phage cocktails with over one thousand individual phage species with hundreds of different strengths (bacteria specificity, host infectivity rates, lysis rates, amount of bacteriophages released upon lysis, etc.) capable of being attached to surfaces in a plethora of applications. It should be appreciated that the resulting phage-modified surfaces disclosed herein can be used in a variety of applications, such as, and without limitation, anti-bacterial surfaces on industrial devices where bacteria could grow, on food processing equipment that could come in contact with bacteria causing food-borne illnesses, as anti-bacterial therapies provided to human patients or animals in need of such therapies, as anti-bacterial surfaces on implanted medical devices as either a prophylactic or therapeutic means, etc. If used for in vivo therapies, the potential size of the bacteriophage population may need to be taken into account and carefully adjusted by increasing or decreasing the covalently bound population of phage(s) on the surface. Another advantage of using covalently attached phages to polymeric surfaces is the ability of in vivo analysis of bacteria and bacterial strength. It should be noted that, in these experiments described herein, only phages covalently attached to polymeric surfaces actively participated in targeted biofilm destruction, as all surface devices were washed at least seven times in PBS buffer to remove all non-covalently bound phage.

Detailed Experiments

Phage Farming

T1 and Φ11 bacteriophages were prepared by Plate lysis method with minor modifications in order to obtain high bacteriophage titer. A heavy suspension of bacteria from a 16 hour incubated plate was suspended in 2 ml of Tryptic Soy Broth (TSB). Then 500 μl of bacterial suspensions, 500 μl of phage stock solution, and 200 μl of cold CaCl₂ at 4° C. were added to a 15 ml Falcon® tube followed by adding 5 ml of top agar (TSB containing 0.75% agar, cooled to 50° C.) and mixing well, then pouring on prepared Tryptic Soy Agar (TSA) plates (pre-warmed for 30 min at 37° C.). The top agar was allowed to cool, and the plates were incubated at room temperature overnight, or until clear lyses of the whole plate were observed. The top agar was scraped gently with a sterile spreader by adding 5-6 ml of PBS. The scraped top agar from all plates was poured into a 50 ml Falcone tube and centrifuged at 10,000 rpm for 10 min at room temperature. The supernatant was collected, and pellet was discarded. The supernatant was filter-sterilized using a 0.45 μm syringe filter with 100 μl of phage filtrate being spread on a plain TSA plate and incubated overnight to ensure sterility.

Phage Purification and Concentration by PEG Method

100 g of PEG (MW 10000) and 6 g of NaCl was mixed with 250 ml of water, autoclaved, and pH adjusted to 7.2 under sterile conditions. One volume of PEG solution was added to four volumes of the bacteriophage supernatant obtained above and refrigerated overnight (stable up to 2 weeks at 4° C.) followed by centrifuging the tubes at 10,000 rpm for 2 hours at 4° C. The supernatant was discarded, and the tube was left in an inverted position for 10-20 min. The pellet was resuspended in 0.1 of the original volume of phage suspension using PBS and stored at 4° C. until needed.

Plaque Formation Assay for the Phage Attached Surfaces

PTFE and PE surfaces exhibiting attached bacteriophages (T1 phage for Escherichia coli and Φ11 phage for Staphylococcus aureus sub spp RN4220) were used for plaque formation assay. In a typical experiment, overnight culture of bacteria (E. coli for T1 phage and S. aureus for Φ11 phage) were diluted 1:1,000 in TSB and allowed to grow for 3 hours. The cells were normalized up to 0.1 (OD₆₀₀ nm). Two 15 ml Falcon® tubes were labeled as T1 and Φ11, and 500 μL of respective bacteria were added. 5 ml of top agar (cooled to 50° C.) was added to each tube and poured into thin layered TSA plates pre-warmed at 37° C. for at least 30 min. Final buffer in which the surface was suspended, the surface without the phage attached to it and phage itself were also included as negative and positive controls. The respective surfaces were then stabbed into the top agar before it solidified. The plates were incubated at room temperature for 24-48 hours, and results were observed in the form of clear plaques seen around the surfaces. The images were taken by Kodak DC 290 and processed via Kodak 1D software. This experiment was performed independently for each type of surface attached with respective phages. For mixed phage attached surfaces, similar assays were performed. Three plates were labeled as T1, Φ, and T1/Φ for each surface. Each mixed phage attached surfaces were tested for plaque formation in these three plates. Buffers in which the surfaces were suspended were also tested to eliminate any free bacteriophages present. Positive and negative controls were included separately in each assay. Images were taken to record the plaques produced by the phages.

Phage Attachment

Medical grade PTFE and UHMWPE (PE) were purchased from McMaster-Carr Supply Co. (Atlanta, Ga.), cut into 1×1 cm squares, washed in isopropanol, and dried at room temperature under vacuum before use. To obtain —COOH terminated PTFE and PE surfaces, microwave plasma reactions were conducted in the presence of maleic anhydride (MA) (Aldrich Chemical Co.) under open reactor conditions, as described elsewhere (see, e.g., Gaboury, S. R., Urban, M. W. Microwave plasma reactions of solid monomers with silicone elastomer surfaces: a spectroscopic study. Langmuir. 9, 3225 (1993)). In the next step, PTFE-COOH and PE-COOH surfaces were placed in PBS buffer pH 7.4 (Invitrogen) containing 2.5 mmol of 1-ethyl-3-(3-dimethylaminopropyl)carbodiimide (EDAC) and 2.5 mmol N-hydroxysuccinimide (NHS) for 2 hours in order to create —COO⁻ groups followed by washing in PBS buffer, then immediate immersion into 10 mL buffer solution containing 500 μL of concentrated T1 phage or Φ11 phage from above for 16 hours. Additional PTFE and PE surfaces were reacted with a 1:1 mixture of T1 and Φ11 phages following the aforementioned process using 500 μL of each phage in 10 mL of PBS buffer. The surfaces were then washed seven times in PBS buffer to remove all non-covalently attached phages.

Characterization

Attenuated total reflectance Fourier transform infrared (ATR FT-IR) spectra were collected using a Bio-Rad FTS-6000 FT-IR single-beam spectrometer set at a 4 cm⁻¹ resolution equipped with DTGS detector and a 45° face angle Ge crystal with a depth of penetration of 0.37 μm Each spectrum represents 200 co-added scans ratioed against a reference spectrum obtained by recording 200 co-added scans of an empty ATR cell. All spectra were corrected for spectral distortions using Q-ATR software.

Atomic force microscopy (AFM) measurements were conducted on either a Nanoscope IIIa Dimension 3000 scanning probe microscope (Digital Instruments) or a Bruker Dimension icon scanning probe microscope with ScanAssist (Digital Instruments). A silicon probe with 125 μm long silicon cantilever, nominal force constant of 40 N/m and resonance frequency of 275 kHz was used in a tapping mode, allowing assessment of surface topography. Quantification of bacteriophages covalently attached to Si surfaces was performed by using ImageJ software (NIH) to analyze surface particles.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

We claim:

1. A method for covalently attaching a bacteriophage to a surface comprising:
a. providing a surface;
b. reacting said surface with a carboxylic acid containing compound to provide carboxylic acid groups covalently attached to said surface;
c. providing at least one bacteriophage; and
d. covalently bonding said at least one bacteriophage to at least one of said carboxylic acid groups on said surface.

2. The method of claim 1, wherein said surface is a silicon wafer.

3. The method of claim 1, wherein said surface is a polymeric surface.

4. The method of claim 3, wherein said polymeric surface is made from an organic polymer.

5. The method of claim 4, wherein said organic polymer is selected from the group consisting of PE and PTFE.

6. The method of claim 1, wherein said carboxylic acid containing compound is maleic acid.

7. The method of claim 6, wherein the step of reacting said surface with a carboxylic acid containing compound to provide carboxylic acid groups covalently attached to said surface includes a microwave plasma reaction in the presence of maleic acid.

8. The method of claim 7, further comprising the step of incubating said surface with carboxylic acid groups covalently attached to said surface with 1-ethyl-3-(3-dimethylaminopropyl)carbodiimide and N-hydroxysuccinimide.

9. The method of claim 1, wherein said at least one bacteriophage is at least one non-enveloped bacteriophage.

10. The method of claim 9, wherein said at least one non-enveloped bacteriophage is a T1 phage.

11. The method of claim 1, wherein said at least one bacteriophage is at least one enveloped bacteriophage.

12. The method of claim 11, wherein said at least one enveloped bacteriophage is a Φ11 phage.

13. The method of claim 1, wherein said at least one bacteriophage is more than one bacteriophage.

14. The method of claim 13, wherein said more than one bacteriophage is selected from the group consisting of an enveloped bacteriophage, a non-enveloped bacteriophage, or combinations thereof.

15. The method of claim 14, wherein said more than one bacteriophage consists of a combination of T1 and Φ11 phages.

16. The method of claim 1, wherein said step of covalently bonding said at least one bacteriophage to at least one of said carboxylic acid groups on said surface comprises the formation of amide linkages between said at least one bacteriophage and said at least one of said carboxylic acid groups.

17. A method for covalently attaching a bacteriophage to a polymeric surface comprising:
a. providing a polymeric surface;
b. reacting said polymeric surface with a carboxylic acid containing compound to provide carboxylic acid groups covalently attached to said polymeric surface;
c. providing at least one bacteriophage; and
d. covalently bonding said at least one bacteriophage to at least one of said carboxylic acid groups on said polymeric surface.

18. The method of claim 17, wherein said polymeric surface is selected from the group consisting of PE and PTFE.

19. The method of claim 17, wherein said carboxylic acid containing compound is maleic acid.

20. The method of claim 19, wherein the step of reacting said surface with a carboxylic acid containing compound to provide carboxylic acid groups covalently attached to said surface includes a microwave plasma reaction in the presence of maleic acid.

21. The method of claim 20, further comprising the step of incubating said surface with carboxylic acid groups covalently attached to said surface with 1-ethyl-3-(3-dimethylaminopropyl)carbodiimide and N-hydroxysuccinimide.

22. The method of claim 17, wherein said at least one bacteriophage is at least one non-enveloped bacteriophage.

23. The method of claim 22, wherein said at least one non-enveloped bacteriophage is a T1 phage.

24. The method of claim 17, wherein said at least one bacteriophage is at least one enveloped bacteriophage.

25. The method of claim 24, wherein said at least one enveloped bacteriophage is a Φ11 phage.

26. The method of claim 17, wherein said at least one bacteriophage is more than one bacteriophage.

27. The method of claim 17, wherein said step of covalently bonding said at least one bacteriophage to at least one of said carboxylic acid groups on said polymeric surface comprises the formation of amide linkages between said at least one bacteriophage and said at least one of said carboxylic acid groups.

28. An antibacterial surface device comprising at least one bacteriophage covalently bound to a surface material, wherein said surface material is reacted with a carboxylic acid containing compound to provide carboxylic acid groups covalently attached to said surface material, and wherein said surface material is selected from the group consisting of silicon wafer and organic polymer.

29. The antibacterial surface device of claim 28, wherein said organic polymer is selected from the group consisting of PE and PTFE.

30. The antibacterial surface device of claim 28, wherein said at least one bacteriophage is covalently bound to said surface material by an amide linkage.

31. The antibacterial surface device of claim 30, wherein said at least one bacteriophage is at least one enveloped bacteriophage.

32. The antibacterial surface device of claim 31, wherein said at least one enveloped bacteriophage is a Φ11 phage.

33. The antibacterial surface device of claim 30, wherein said at least one bacteriophage is more than one bacteriophage.

34. The antibacterial surface device of claim 33, wherein said more than one bacteriophage is selected from the group consisting of an enveloped bacteriophage, a non-enveloped bacteriophage, or combinations thereof.

35. The antibacterial surface device of claim 34, wherein said more than one bacteriophage consists of a combination of T1 and Φ11 phages.
